(19) United States
(12) Patent Application Publication
(10) Pub. No.: US 2016/0361191 A1
(43) Pub. Date: Dec. 15, 2016

(54) Male Incontinence Device

(71) Applicant: Dai-Vi Moon, Chattanooga, TN (US)

(72) Inventor: Dai-Vi Moon, Chattanooga, TN (US)

(21) Appl. No.: 15/045,747

(22) Filed: Feb. 17, 2016

Related U.S. Application Data
(60) Provisional application No. 62/174,616, filed on Jun. 12, 2015.

(51) Int. Cl. A61F 5/453; A61F 13/84; A61F 13/58

(52) U.S. Cl. CPC A61F 5/453; A61F 13/58; A61F 13/84; A61F 2013/15121

(57) ABSTRACT

An incontinence device adapted for male use. The incontinence device has a base having an interior side and an exterior side. The interior side of the base includes an absorbent layer attached to a center portion of the base. The interior side further includes an adhesive layer disposed thereon. In use, the absorbent layer forms a pocket configured to secure a penis therein. A waterproof layer is disposed between the adhesive layer and the absorbent layer. In some embodiments, the incontinence device further includes an alarm mechanism that is configured to notify the wearer to check the incontinence pad for urine or other discharge. One embodiment of the incontinence device further includes a plug insertable into the user's urethra in order to prevent accidental discharge of urine or other liquids.

### CROSS REFERENCE TO RELATED APPLICATIONS

This application claims the benefit of U.S. Provisional Application No. 62/174,616 filed on Jun. 12, 2015. The above identified patent application is herein incorporated by reference in its entirety to provide continuity of disclosure.

### BACKGROUND OF THE INVENTION

The present invention relates to absorbent devices. More particularly, the present invention relates to devices for absorbing urine and other discharge resulting from incontinence.

Many individuals struggle with incontinence on a daily basis. Urinary incontinence may be caused by medical procedures, such as urinary tract surgeries or bladder surgeries. Others may have a physical or mental handicap that causes incontinence. Aging is also another cause of incontinence. Male individuals in particular also suffer from incontinence related to prostate issues. Those suffering from incontinence may lose control of their bladder as a result of various actions as coughing, sneezing, and lifting objects. In addition to causing embarrassment, incontinence can cause discomfort and infection if skin contacts discharged urine for an extended period.

Devices that are designed to alleviate the inconveniences of incontinence have been developed. These devices, however, suffer from various deficiencies. Most incontinence devices consist of garments that are securable around and under a user's waist. These can be difficult to wrap and secure in such a way that no liquid is permitted to leak through to the wearer's skin. Other incontinence devices are bulky undergarments that are often noticeable through the wearer's outer clothing. These devices are difficult to conceal under clothing and may potentially cause embarrassment to the wearer. Also, these devices typically lack an additional space or securement means for the penis, which is desirable for male users. It is therefore desirable to provide a male incontinence device that is securable to the tip of a user's penis in order to absorb the discharge of urine and other liquids therefrom.

### SUMMARY OF THE INVENTION

In view of the foregoing disadvantages inherent in the known types of incontinence devices now present in the prior art, the present invention provides a male incontinence device wherein the same can be utilized for providing convenience for the user when alleviating the symptoms of incontinence. The incontinence pad comprises a base having an interior side and an exterior side, an absorbent layer disposed on a center portion of the interior side, an adhesive layer surrounding the absorbent layer disposed on a second portion of the interior side configured to secure the device to a user's penis, and a waterproof layer disposed between the absorbent layer and the adhesive layer.

One object of the present invention is to provide a male incontinence device that has all of the advantages of prior art devices and none of the disadvantages.

Another object of the present invention is to provide a male incontinence device that may be worn discretely and that permits freedom of movement for the wearer.

A further object of the present invention is to provide an incontinence device adapted to prevent male genitalia from coming into contact with discharged urine.

Yet another object of the present invention is to provide an incontinence device capable of absorbing moisture and further facilitating the evaporation thereof.

Still a further object of the present invention is provide an incontinence device having a plug secured to a post extending upwardly from an absorbent layer, wherein the plug is insertable into an individual's urethra in order to prevent the discharge of urine therefrom.

Still another object of the present invention is to provide an incontinence device having an alarm mechanism configured to notify the wearer at predetermined intervals to check the incontinence device for discharge and change the incontinence device if necessary.

Other objects and features of the present invention will be apparent from the following description.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an incontinence device according to the present invention.

FIG. 2 shows a perspective view of an incontinence device according to the present invention being attached to a user's penis.

FIG. 3 shows a perspective view of an embodiment of an incontinence device according to the present invention having an alarm mechanism.

FIG. 4 shows a perspective view of an incontinence device according to the present invention secured to a user's penis.

FIG. 5 shows a perspective view of an alternate embodiment of an incontinence device according to the present invention.

### DETAILED DESCRIPTION OF THE INVENTION

Reference is made herein to the attached drawings. Like reference numerals are used throughout the drawings to depict like or similar elements of the incontinence device. For the purposes of presenting a brief and clear description of the present invention, the preferred embodiment will be discussed as used for a wearer suffering from incontinence. The figures are intended for representative purposes only and should not be considered to be limiting in any respect.

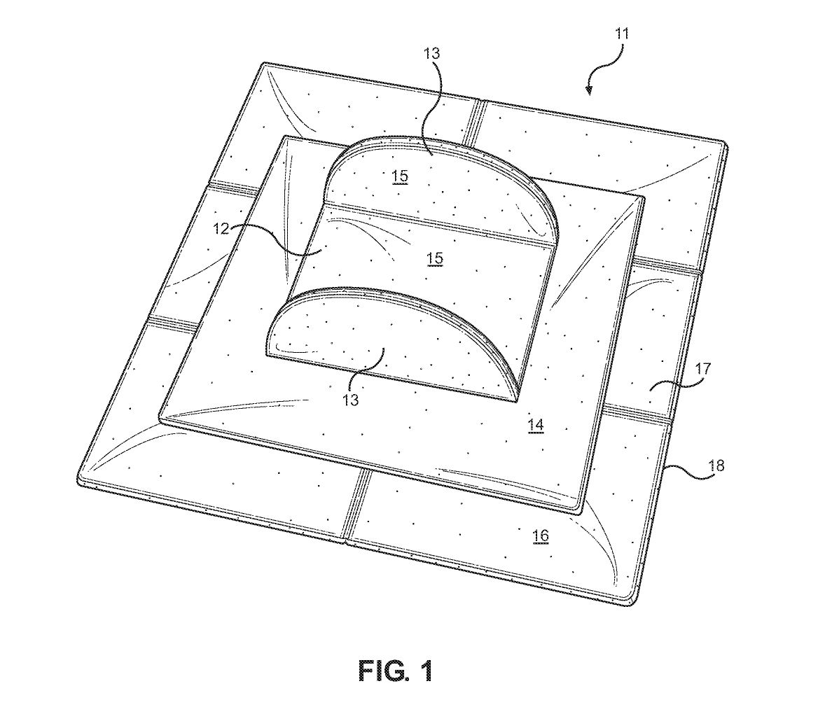

Referring now to FIG. 1, there is shown a perspective view of an incontinence device according to the present invention. The incontinence device 11 is adapted for use by a male individual who suffers from urinary or other forms of incontinence and is securable to a user's penis in order to absorb any discharge of urine therefrom. The incontinence device 11 generally comprises a base 18 having an interior side 17 and an exterior side (not shown). The base 18 may be made in various shapes and sizes in order to accommodate different users. The materials that form the base 18 are preferably vapor permeable so that the incontinence device 11 is breathable when worn, which provides comfort for the user.

An absorbent layer 15 having perimeter edges 13 is disposed on a center portion 12 of the interior side 17. The absorbent layer 15 contacts the tip of a user's penis when the incontinence device 11 is in use. The absorbent layer 15 may be made of any material capable of absorbing urine or other types of discharge and retaining the discharge under pressure experienced during use. An adhesive layer 16 is disposed on the interior side 17 of the base surrounding the perimeter edges 13 of the absorbent layer 15. The adhesive layer 16 may comprise any pressure-sensitive adhesive material adapted to secure an object to skin. The adhesive layer 16 secures the incontinence device 11 to a user's penis during use. Further, a waterproof layer 14 is disposed on the interior side 17 of the base 18 and is further disposed between the absorbent pad 15 and the adhesive layer 16. The waterproof layer 14 is liquid impermeable and prevents discharged urine not immediately absorbed by the absorbent layer 15 from leaking through the exterior side of the incontinence device 11.

The shape of the absorbent layer 15 is defined by the absorbent layer periphery edges 13. In one embodiment of the invention, one or more of the periphery edges 13 extend orthogonally from the base 18. The orthogonal periphery edges 13 provide additional support structure to the urinary incontinence device when in use. However, flat periphery edges 13 may also be utilized in order to reduce the overall size of the incontinence device 11. The periphery edges 13 may further be used to determine the placement of a wearer's penis when attaching the incontinence device 11 thereto.

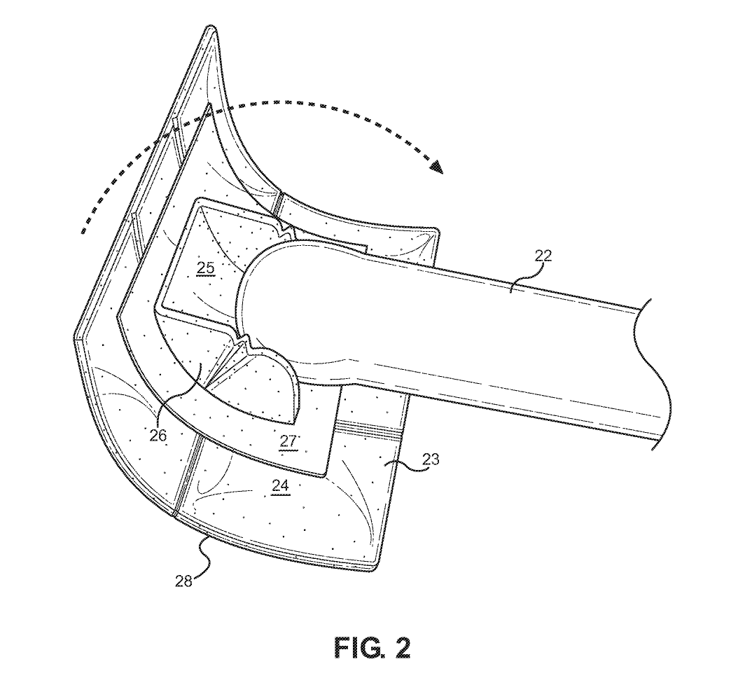

Referring now to FIG. 2, a perspective view of an incontinence device according to the present invention being attached to a user's penis is shown. The interior side 23 of the base 28 faces the user during use. The absorbent layer 25 contacts the tip of the user's penis 22 during use and the base 28 is then folded over. The folded absorbent layer 25 then forms a pocket 26 around the tip of the user's penis 22. The absorbent layer 25 absorbs any material discharged from the penis 22 while the waterproof layer 27 prevents the discharged material from interfering and possibly compromising the effectiveness of the adhesive layer 24.

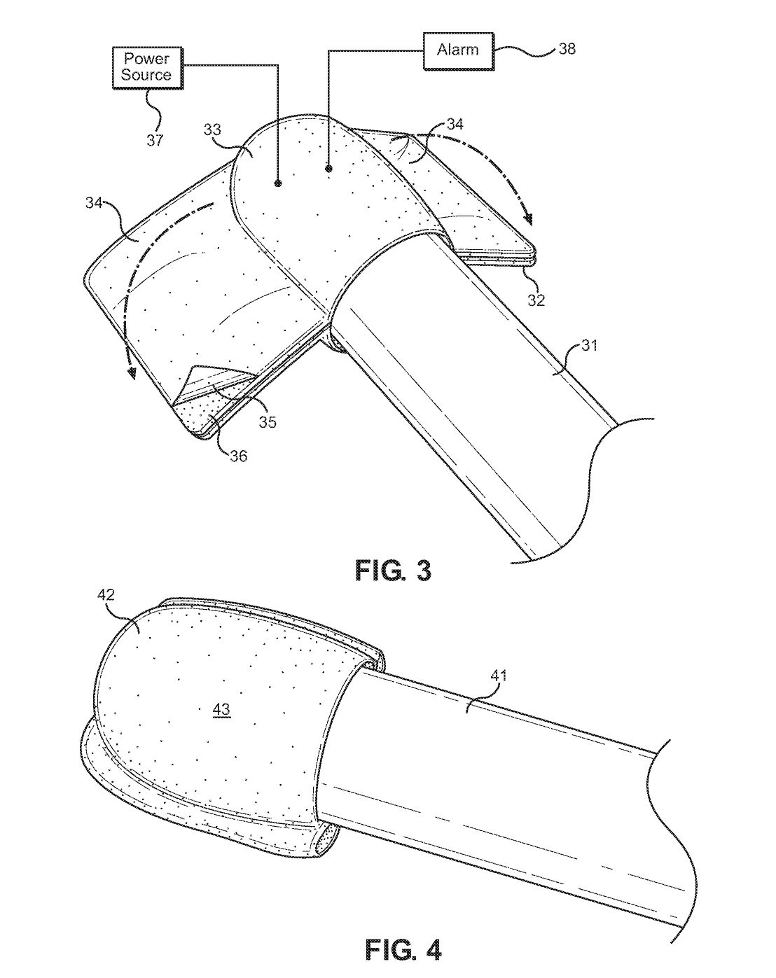

Referring now to FIG. 3, a perspective view of an embodiment of an incontinence device according to the present invention having an alarm mechanism is shown. In one embodiment of the invention, the incontinence device further comprises an alarm mechanism 38 disposed in an interior volume formed between the interior side and exterior side of the base. A power source 37 such as a battery is also disposed between the interior side and exterior side of the base. The power source 37 is operatively connected to the alarm mechanism 38 in order to provide power thereto. The alarm mechanism 38 may comprise a small vibration motor configured to vibrate at predetermined time intervals in order to remind the user to check the incontinence pad for discharge and replace the incontinence pad if necessary. Further, the incontinence device may comprise a transparent film 35 disposed over either one of or both the interior side (not shown) and exterior side 36 of the base. The transparent film 35 allows the device to remain sterilized and may be peeled off prior to use.

Referring now to FIG. 4, a perspective view of an incontinence device according to the present invention secured to a user's penis is shown. The tip of the user's penis 41 is secured within the pocket 42 formed by the absorbent layer. The base portion is then folded over the penis such that the exterior side 43 forms a contiguous surface with the penis 41. Once the base has been folded over the penis, pocket 43 encloses the penis within the absorbent layer. The outer side comprises a water impermeable material disposed thereon. The water impermeable material may be any material adapted to prevent moisture from passing through the outer side of the incontinence device.

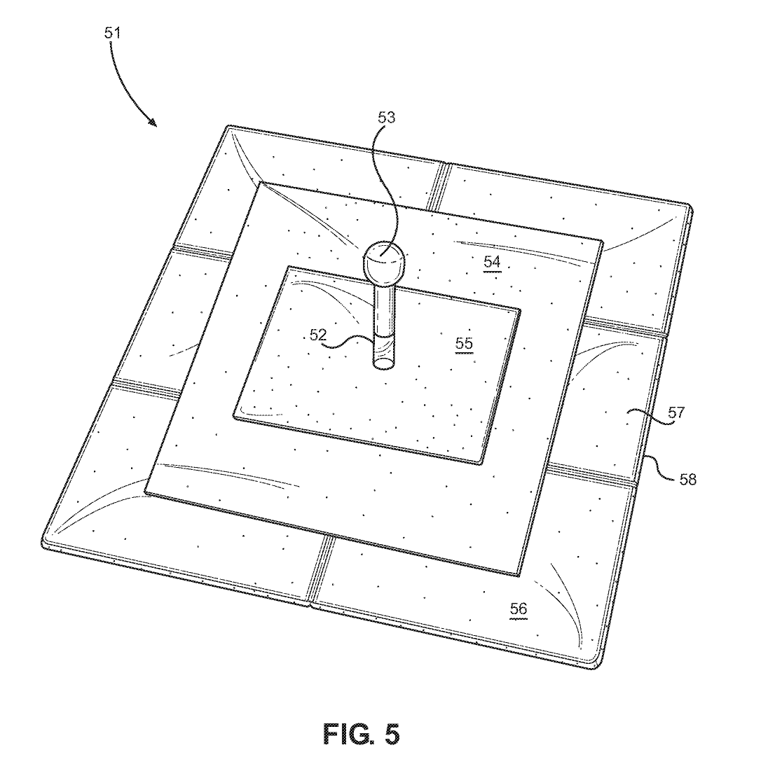

Referring now to FIG. 5, a perspective view of an alternate embodiment of an incontinence device according to the present invention is shown. The illustrated embodiment of the incontinence device 51 further comprises a post 52 attached at one end to the absorbent layer 55, extending from the absorbent layer 55 orthogonal to the base 58. A plug 53 is secured to the free end of the post 52. The plug 53 is insertable into a user's urethra in order to prevent the discharge of fluid therefrom. The post 52 may be hollow in order to cause liquids not blocked by the plug 53 to be redirected through the post 52 and to the absorbent layer 55. The plug 53 and post 52 may comprise any suitable material such as an expandable foam or medical grade rubber, respectively. The combination of the plug 53 with the waterproof layer 54 and absorbent layer 55 provides increased liquid blocking properties to the incontinence device 51.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) An incontinence device, comprising:
a base comprising an interior side and an exterior side;
a waterproof layer disposed on a first portion of the interior side of the base;
an absorbent layer disposed on a center portion of the waterproof layer comprising one or more periphery edges attached to a center portion of the interior side;
an adhesive layer disposed on a second portion of the interior side, wherein the adhesive layer surrounds said one or more periphery edges of said absorbent pad;

2) The incontinence device of claim 1, wherein one or more of the absorbent layer periphery edges extend orthogonally from the interior side of the base.

3) The incontinence device of claim 1, wherein the interior side of the base further comprises a removable transparent film disposed on an uppermost surface thereof.

4) The incontinence device of claim 1, wherein the exterior side of the base further comprises a removable transparent film disposed on an uppermost surface thereof.

5) The incontinence device of claim 1, wherein the incontinence device is adapted to be used by a male user.

6) The incontinence device of claim 1, wherein securing the base to a user's penis via the adhesive layer causes the absorbent layer to form a pocket configured to secure a penis therein, the pocket comprising a closed end having peripheral sidewalls extending therefrom, and an open end configured to receive a penis therethrough.

7) The incontinence device of claim 1, wherein the base further comprises an interior volume disposed between the interior side and the exterior side thereof, a power source disposed within the interior volume, and an alarm mechanism disposed within the interior volume operatively connected to the power source.

8) The incontinence device of claim 7, wherein the alarm mechanism comprises a vibration motor that is configured to vibrate at a predetermined time interval.

9) The incontinence device of claim 7, wherein the power source is a battery.

10) The incontinence device of claim 1, further comprising:
a post extending orthogonally from a center portion of the absorbent pad;
a plug secured to the free end of the post;
wherein said plug is insertable into an individual's urethra.

11) The incontinence device of claim 9, wherein said post comprises peripheral sidewalls that surround an interior channel, wherein said interior channel is configured to direct moisture to the absorbent pad.
